(19) United States
(12) Patent Application Publication
(10) Pub. No.: US 2011/0288320 A1
(43) Pub. Date: Nov. 24, 2011

(54) PURIFICATION TREATMENT OF FATTY MATERIALS

(76) Inventors: Massoud Jalalpoor, Schifferstadt (DE); Patrick Brems, Worms (DE); Wim DeGreyt, Sinaai (BE); Marc Kellens, Mechelen-Muizen (BE)

(21) Appl. No.: 12/743,726

(22) PCT Filed: Nov. 27, 2008
(86) PCT No.: PCT/EP08/10044

§ 371 Date: Apr. 21, 2011

Related U.S. Application Data
(60) Provisional application No. 61/004,349, filed on Nov. 27, 2007.

(51) Int. Cl. C11B 3/10; B01D 17/00; B01D 11/04; C07C 57/12; B01J 19/00

(52) U.S. Cl. 554/191; 554/223; 252/182.12; 210/511; 210/202

(57) ABSTRACT

A process for the removal of impurities from fatty materials comprising the steps of providing a fatty material that may already have undergone some purification treatment, adding an aqueous suspension of liquid adsorbent to the fatty material and causing it to form a mixture with the fatty material, optionally adding some water to the mixture, and forming an aqueous phase separate from the fatty material and removing the aqueous phase from the fatty material.

### FIELD OF THE INVENTION

The invention relates to the removal of residual impurities from fatty materials by an adsorptive treatment with liquid adsorbent.

### BACKGROUND OF THE INVENTION

In the context of the present invention, fatty materials are defined as products that consist mainly of organic molecules comprising fatty acid moieties. In particular, they refer to products that have already undergone some kind of purification treatment or chemical modification but still contain residual impurities and therefore need further purification treatment.

Edible oils and fats as produced by the expelling and/or extraction of oilseeds or by the rendering of fatty animal tissue contain many different impurities. With only a few exceptions such as virgin olive oil and some specialty oils, they have to be purified before they can be sold to the general public or used in the manufacture of food or oleochemical products, and even such oleochemical products may require the removal of further residual impurities to ensure that they perform properly.

In the edible oil industry, a number of different purification processes are being used. They comprise the degumming of crude oils to remove some or most phosphatides and other, water-soluble compounds. Degummed oil, a fatty material in the context of the present invention, can then be alkali refined to remove free fatty acids, the alkali refined neutral oil, also a fatty material in the context of the present invention, can be bleached to remove colouring compounds and since the bleaching process involves a filtration step, it also removes particulate matter. Finally, the bleached oil can be deodorised in a vacuum steam stripping process to remove malodorous compounds.

In the oleochemical industry, fatty acid methyl esters (FAME) that should also be considered as a fatty material in the context of the present invention, can be washed with water to remove residual methanol and glycerol and other, water-soluble impurities. However washing with water may not remove water-insoluble impurities so that another purification treatment may be desirable.

The purification treatments mentioned above have in common that they remove the bulk of the impurities but leave some residual impurities in the fatty material. The water degumming process for instance only removes hydratable phosphatides and leaves the non-hydratable phosphatides (NHP) in the oil. To remove these NHP, a degumming acid has to be used. This converts the NHP into hydratable phosphatidic acid that can be removed as an aqueous sludge especially when the pH is raised to such an extent that the phosphatidic acid is dissociated. However, as shown in U.S. Pat. No. 4,469,185, removing the phosphatidic acid as an aqueous sludge leaves some residual phosphatides in the oil necessitating a subsequent water wash.

Similarly, oil from which a soapstock has been removed during the alkali refining treatment or neutralisation treatment still contains residual soaps that have to be removed from the oil because they interfere with the subsequent bleaching treatment. They can be removed by two successive water-washing treatments but this has the disadvantage of creating aqueous effluent that requires appropriate treatment before it can be disposed of. Accordingly, U.S. Pat. No. 4,629,588 discloses a method for refining glyceride oils using amorphous silica with effect pore diameters of about 60 to 5000 Ångstroms for the removal of trace contaminants specifically phospholipids from glyceride oils. This latter treatment avoids the aqueous effluent but generates a solid waste product instead.

Another alkali refining process employing a form of silica has been disclosed in British Patent 599,595. This process of refining a crude glyceride oil comprises mist-mixing the oil with a water-adsorptive agent selected from the group consisting of salts containing water-adsorptive anions, “colloidal silica,” and mixtures thereof, and with an aqueous caustic alkali solution which acts to neutralize the free fatty acids present in the oil to form soapstock. The soapstock is separated from the oil, the caustic alkali being used in an amount in such excess of that necessary to neutralize the free fatty acids that some of the caustic alkali is present at the time of separation. The caustic is used in an amount effective to substantially reduce the neutral oil retained by the soapstock and to retard the saponification of the neutral oil. In this process, “colloidal silica” is not added to the oil, only sodium silicate and under the conditions that the silicate is added, “colloidal silica” will not be formed. After the soapstock removal, the oil can be cooled to cause residual soapstock to agglomerate. Subsequently, the agglomerated soapstock particles can then be removed by a clarifying centrifuge.

U.S. Pat. No. 6,027,755 discloses a bleaching product manufactured by agglomerating bleaching clay particles with a particle size distribution wherein 95% of the particles are from about 0.2 to 10 microns by the use of a binder that may be colloidal clay having an average particle size less than one-half micron. After agglomeration, the resulting microspheres have an average diameter of at least 10 microns, preferably about 20 microns to 30 microns.

In the production of FAME for use as a biodiesel, triglyceride oil is allowed to react with methanol in the presence of an alkaline catalyst such as an alkali metal hydroxide or methoxide. This reaction causes glycerol to be formed and since glycerol is hardly soluble in the FAME, the reaction mixture splits into two separate phases. Again this separation is not complete and accordingly, the excess methanol is divided over both phases and some glycerol is dissolved in the FAME phase. Moreover, the inactivation of the alkaline catalyst leads to salt formation which salts are also divided over the two phases. Consequently, the FAME phase, also being considered as a fatty material in the context of the present invention, still needs further purification.

In addition, the FAME phase often comprises unwanted compounds causing haze and/or having detrimental effect on cold soak test and on the total contamination. One of the major components involved in the development of haze are the free sterol glucosides. In fact, most of the triglyceride oil used for the production of FAME contains acyl sterol glucosides, but the reaction conditions during the transesterification are such that they will cause the acyl group in the sterol glucosides to be transesterified under formation of FAME and free sterol glucosides. This latter compound is poorly soluble in FAME and even low concentration of it may crystallise and form a haze. When using the FAME as a diesel fuel component, this haze is most undesirable since it will clog fuel filters and eventually interrupt the fuel supply. Of course, in addition to sterol glucosides, other residual impurities may also contribute to the haze formation, further increase the total contamination and cause cold soak test failure. Accordingly, the FAME used for biodiesel also needs highly efficient purification.

Accordingly it is an object of the invention to overcome the various disadvantages of the prior art by using liquid adsorbent in the processing of fatty materials.

It is an object of the invention to eliminate a filtration step during the processing of fatty materials.

It is a further object of the invention to reduce the usage of process water and production of wastewater during the processing of fatty materials.

It is also an object of the invention to provide a biodiesel that does not develop a haze on storage.

These and further objects of the invention will become apparent from the description and examples hereinafter.

### SUMMARY OF THE INVENTION

It has surprisingly been found that liquid adsorbent is capable of removing impurities such as but not limited to phosphatides, soaps and sterol glucosides from various fatty materials in a process comprising the steps of:
a) providing a fatty material that may already have undergone some purification treatment;
b) adding an aqueous suspension of liquid adsorbent to said fatty material and causing it to form a mixture with said fatty material;
c) optionally adding some water to said mixture;
d) forming an aqueous phase that is separate from the fatty material and removing the aqueous phase from the fatty material.

Consequently, the process of the invention may be profitably used in various processes that precede and involve the production and/or the purification of biodiesel.

Another embodiment of the present inventions relates to a fatty material composition including, fatty material that may already have undergone some purification treatment, liquid adsorbent, and water.

A further embodiment of the present invention relates to an apparatus suitable for removing impurities from fatty material including, a mixing device that is suitable for mixing liquid adsorbent with the fatty material; and a separating device in-line with the mixing device that is suitable for removing the liquid adsorbent from the fatty material.

An even further embodiment of the present invention regards an apparatus suitable for removing impurities from fatty material including, a first mixing device that is suitable for mixing the fatty material with an acid to form solid impurities and fatty material, a first separating device in-line with the first mixing device that is suitable for removing the solid impurities from the fatty material, a second mixing device in-line with the first separating device that is suitable for mixing liquid adsorbent with the fatty material, and a second separating device in-line with the second mixing device that is suitable for removing the liquid adsorbent from the fatty material.

### BRIEF DESCRIPTION OF THE DRAWINGS

FIG. 1 illustrates a fatty material refining process according to conventional practice.

FIG. 2 illustrates a fatty material refining process according to conventional practice.

FIG. 3 illustrates a fatty material refining process according to the present invention.

FIG. 4 illustrates a fatty material refining process according to the present invention.

### DETAILED DESCRIPTION OF THE INVENTION

As utilized herein, “acid degumming” is a degumming process in which crude oil is treated with a strong acid to decompose the non-hydratable phosphatides present in the crude oil and thereby liberate phosphatidic acid. This phosphatidic acid is then hydrated by the addition of water so that it can be separated from the degummed oil.

As used herein, “cold soak test” is the time in seconds it takes for cold soaked biodiesel to pass through two 0.8 micron filters and the amount of particulate matter expressed in mg/l collected on the filter as per ASTM 6217.

As used herein “acid refining” is a degumming process in which crude oil is treated with a strong degumming acid to decompose the non-hydratable phosphatides. This phosphatidic acid is then hydrated when said degumming acid is partially neutralised by the addition of a base so that it can be separated from the degummed oil.

As utilized herein, “crude oil” is the general name for a fatty material as isolated from its source and that has not undergone any treatment except perhaps a water degumming treatment ensuring that the crude oil meets trading specifications and does not throw a deposit during storage and transport. Crude oil therefore may contain free fatty acids and/or gums.

As used herein, “degumming” is the general term for the removal of phosphatides from a crude oil by washing it with an aqueous solution (water degumming), by treating it with an acid solution (acid degumming) followed by water washing, or treating it with an acid solution followed by partial neutralisation (acid refining).

As utilized herein, “FAAE” is the abbreviation for Fatty Acid Alkyl Esters and “FAME” is the abbreviation of Fatty Acid Methyl Esters.

As used herein, “fatty material” is defined as products derived from plant or animal material that consist mainly of organic molecules comprising fatty acid moieties. This definition of “fatty material” includes explicitly oleochemical derivatives of oils and fats, such as fatty acid esters of lower alcohol.

As utilized herein, “FFA” is the standard abbreviation of Free Fatty Acids.

As used herein, “metal oxides” is defined as binary oxygen compounds where the metal is the cation and the oxide is the anion. The metals may also include metalloids. Metals include those elements on the left of the diagonal line drawn from boron to polonium on the periodic table. Metalloids or semi-metals include those elements that are on this line. Examples of metal oxides include silica, alumina, titania, zirconia, etc., and mixtures thereof.

As used herein, “liquid adsorbent” is a material that is in a continuous liquid phase and that is capable of refining crude oil, including but not limited to, sols, colloids, suspensions, and the like, and mixtures thereof.

As used herein, “separating device” includes filters, centrifuges, clarifier, decanters and the like.

As utilized herein, “soapstock” is the by-product of the chemical neutralisation of crude triglyceride oils. It comprises soaps, phosphatides and neutral oil besides many colouring compounds, particulate matter and other impurities as well as water containing various salts.

As utilized herein, “transesterification” is another name for alcoholysis. It is the reaction between an alcohol and a glyceride such as an oil or fat. If the alcohol is methanol, alcoholysis can also be referred to as methanolysis.

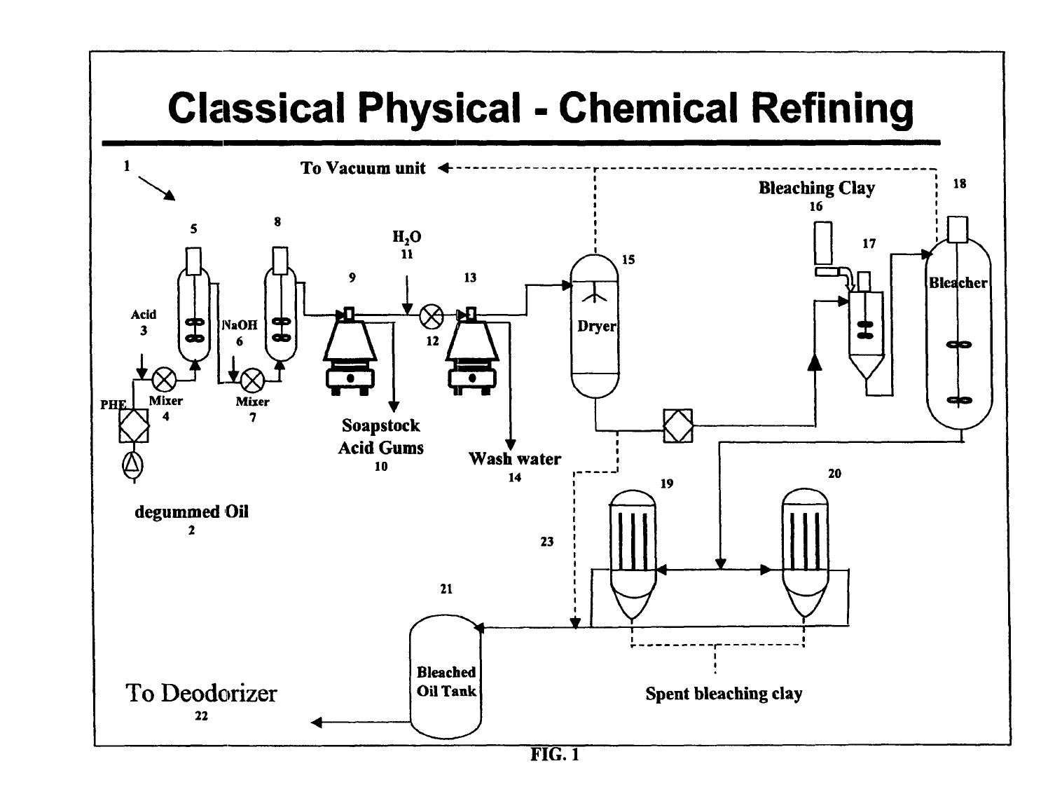

FIG. 1 depicts a conventional apparatus 1 for the physical or chemical refining of fatty material, such as edible oil. Degummed oil 2 is mixed with an acid 3 using an in-line mixer 4, which is then fed into tank 5 and subsequently mixed thoroughly (e.g., for about 2 to about 5 minutes). Then this mixture is combined with caustic 6 with an in-line mixer 7. Subsequently, the mixture is fed into a mixing tank 8 where it is mixed for a period of time suitable to form gums in an aqueous phase (e.g., about 15 to about 30 minutes). This mixture is then sent to a first centrifuge 9 where the gums 10 are separated from the oil and then discarded. The oil is then washed with water 11 using an in-line mixer 12 and sent to a second centrifuge 13 where the aqueous phase 14 is separated from the oil. Subsequently, the oil is sent to a dryer 15 and then combined with an adsorbent or bleaching clay 16 in a high shear mixing tank 17 to remove impurities (e.g., phospholipids, and associated trace elements such as Ca, Mg and Fe) from the oil. The mixture is then sent to a bleacher 18, followed by separation of the adsorbent from the oil using filters 19 and 20. The oil is then optionally stored in a holding tank 21 or subjected to deodorization 22 to remove free fatty acids. For biodiesel production, the oil from dryer 15 may be sent directly to tank 21 via line 23 without the additional bleaching treatment.

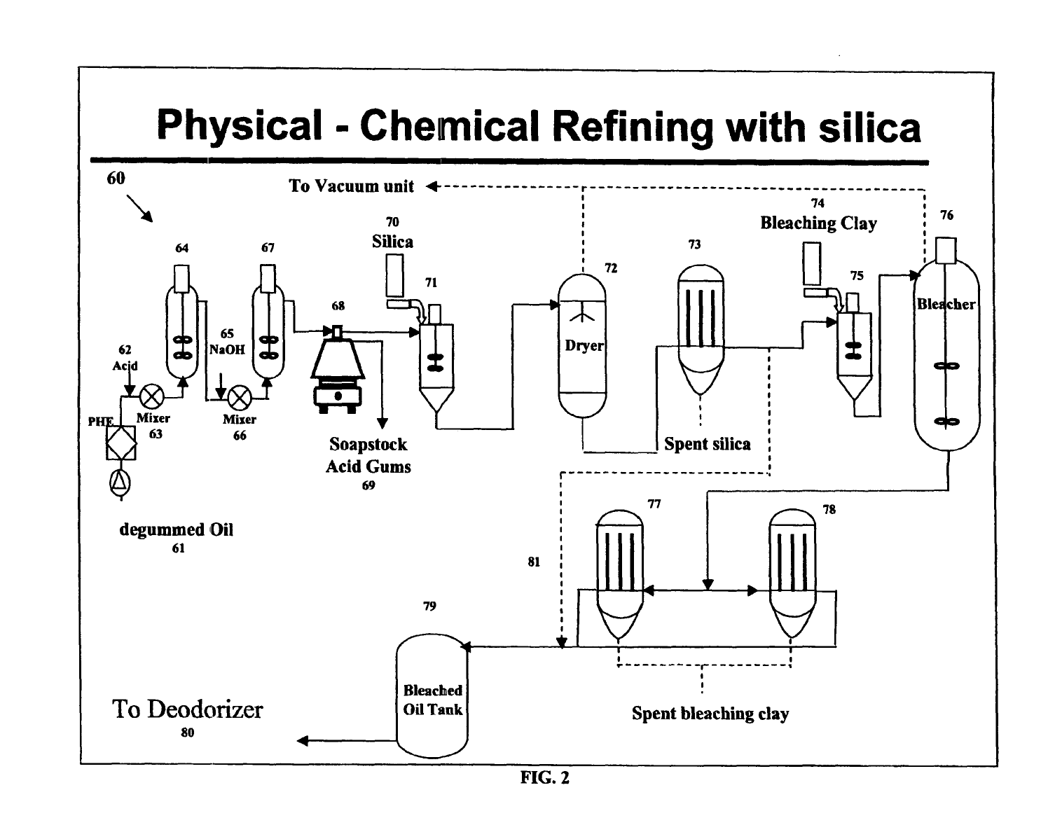

FIG. 2 depicts a conventional apparatus 60 for the physical or chemical refining of fatty material, such as edible oil. Degummed oil 61 is mixed with an acid 62 using an in-line mixer 63, which is then fed into tank 64 and subsequently mixed thoroughly (e.g., for about 2 to about 5 minutes). Then this mixture is combined with caustic 65 with an in-line mixer 66. Subsequently, the mixture is fed into a mixing tank 67 where it is mixed for a period of time suitable to form gums in an aqueous phase (e.g., about 15 to about 30 minutes). This mixture is then sent to a centrifuge 68 where the gums 69 are separated from the oil and then discarded. The oil is then combined with an adsorbent 70, such as TriSyl® silica available from Grace GmbH & Co. KG, using a mixing tank 71 in order to remove impurities (e.g., phospholipids, associated trace elements such as Ca, Mg and Fe, and free fatty acids) from the oil. Subsequently, the oil is sent to a dryer 72 and then the adsorbent is removed from the oil using a filter 73. The oil is then combined with a bleaching clay 74 using mixing tank 75 and the mixture is sent to a bleacher 76, followed by separation of the clay from the oil using filters 77 and 78. The oil is then optionally stored in a holding tank 79 and subjected to deodorization 80 to remove free fatty acids. For biodiesel production, the oil from filter 73 may be sent directly to tank 79 or directly to deodorization 80 via line 81 without the additional bleaching treatment.

The present invention eliminates several of the purification steps utilized in conventional oil refining and degumming processes. In one embodiment of the invention, a fatty material is provided that needs further purification, for instance because previous separation steps have left some impurities in said fatty material. In a first embodiment of the process according to the invention, the fatty material is a degummed triglyceride oil which therefore still contains free fatty acids. Said fatty material may have been acid degummed or acid refined by first of all dispersing a degumming acid into the fatty material and thereby decomposing the non-hydratable phosphatides present in the fatty material, and subsequently raising the pH of the aqueous phase comprising the degumming acid by dispersing water or a base such as, e.g., caustic soda into said acid-fatty material dispersion. These treatments cause the gums to form a separate phase that can be removed by centrifuge.

Because the above-mentioned removal step does not completely remove all of the impurities in the fatty material, further purification steps such as a subsequent water washing as disclosed in U.S. Pat. No. 4,698,185 or a two-centrifuge process with recycling as disclosed in EP 0 349 718 are typically necessary. Instead of using these further conventional purification steps as mentioned above, a process according to the invention may be used to remove residual phosphatides from the degummed triglyceride oil (e.g. the acid degummed or acid refined oil). Accordingly, the oil leaving the centrifugal separator used to remove the gums from the acid refined oil may be treated according to a process of the invention by mixing it with an aqueous liquid adsorbent. There is no need to change its temperature, which will be in the range of 80° C. to 100° C., but the use of lower temperatures is also within the scope of the present invention.

A further embodiment of the present invention relates to an apparatus suitable for removing impurities from fatty material including, a mixing device that is suitable for mixing liquid adsorbent with the fatty material; and a separating device in-line with the mixing device that is suitable for removing the liquid adsorbent from the fatty material.

An even further embodiment of the present invention regards an apparatus suitable for removing impurities from fatty material including, a first mixing device that is suitable for mixing the fatty material with an acid to form solid impurities and fatty material, a first separating device in-line with the first mixing device that is suitable for removing the solid impurities from the fatty material, a second mixing device in-line with the first separating device that is suitable for mixing liquid adsorbent with the fatty material, and a second separating device in-line with the second mixing device that is suitable for removing the liquid adsorbent from the fatty material. The apparatus may include a conduit connecting an outlet of the second separating device to an inlet of the first separating device that is suitable for recycling the liquid adsorbent from the second separating device to the first separating device.

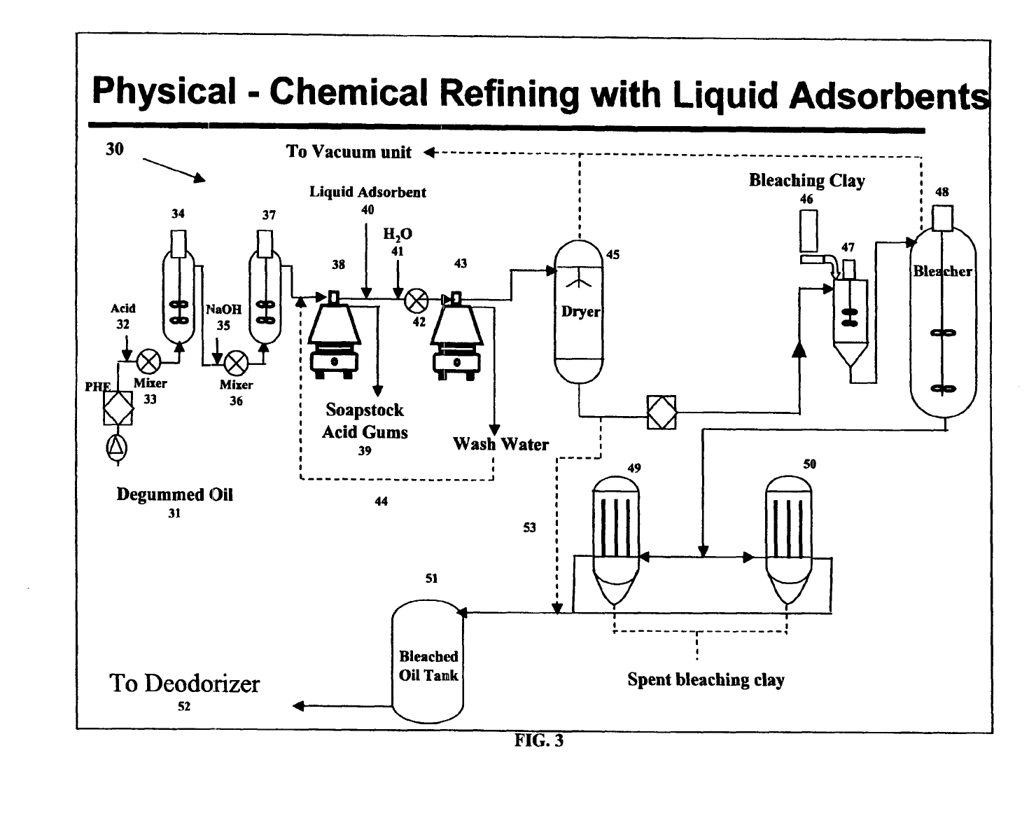

FIG. 3 depicts an apparatus 30 according to the present invention for the physical or chemical refining of fatty material, such as edible oil. Degummed oil 31 is mixed with an acid 32 using an in-line mixer 33, which is then fed into tank 34 and subsequently mixed thoroughly (e.g., for abut 2 to about 5 minutes). Then this mixture is combined with caustic 35 with an in-line mixer 36. Subsequently, the mixture may be fed into a mixing tank 37 where it is mixed for a period of time suitable to form gums in an aqueous phase (e.g., about 15 to about 30 minutes). This mixture is then sent to a first centrifuge 38 where the gums 39 are separated from the oil and then discarded. The oil may then be combined with liquid adsorbent 40 and water 41 using an in-line mixer 42 and sent to a second centrifuge 43 where the aqueous phase 44 is separated from the oil. The aqueous phase includes the liquid adsorbent. Even though the liquid adsorbent 40 is preferably added to the oil after the first centrifuge 38, it may also be added anytime prior to it. In addition, the aqueous phase 44 may be recycled back to the oil stream prior to the first centrifuge 38. Moreover, even though this embodiment includes two centrifuges, the process may be conducted with only one centrifuge, wherein the liquid adsorbent is added prior to this centrifuge. Additionally, the process may also be conducted with more than two centrifuges. Subsequent to the centrifuge(s), the oil may be sent to a dryer 45 and then combined with an adsorbent 46 in a high shear mixing tank 47 to remove impurities (e.g., phospholipids, associated trace elements such as Ca, Mg and Fe, and free fatty acids) from the oil. The mixture may then be sent to a bleacher 48, followed by separation of the adsorbent from the oil using filters 49 and 50. The oil is then optionally stored in a holding tank 51 or subjected to deodorization 52 to remove free fatty acids. For biodiesel production, the oil from dryer 45 may be sent directly to tank 51 via line 53 without the additional adsorbent treatment.

In a second embodiment of the process according to the present invention, the fatty material to be purified is a triglyceride oil that has been subjected to an alkali neutralisation process but that has not yet been washed with water. Such alkaline refined triglyceride oil may contain some residual soaps, phosphatides and water that may profitably be removed by a process according to an embodiment of the present invention whereby the oil is treated with an aqueous liquid adsorbent. Again, there is no need to change its temperature, which will be in the range of 80° C. to 95° C. However, the use of lower temperatures is within the scope of the present invention.

In another embodiment of the present invention, the material to be purified is a fatty material comprising esters of lower alkanols, e.g., methanol and fatty acids, such as the product resulting from a transesterification process employing e.g. methanol as a monohydric alcohol. After the transesterification reaction, the reaction mixture is separated into two phases: a heavy phase comprising glycerol, the lower alcohol, e.g. methanol, and part of the transesterification catalyst, and a lighter phase comprising the fatty acid esters, the lower alcohol, some traces of glycerol and a small portion of the catalyst. Commonly, the lower alcohol, e.g., methanol, is removed from the lighter phase by evaporation but the evaporation residue still contains traces of alcohol, traces of glycerol and part of the transesterification catalyst, which components are commonly removed by water washing.

According to an embodiment of the process of the invention, liquid adsorbent may be added to the washing water and optionally, the water comprises enough acid (e.g. citric acid) to neutralise the residues of the catalyst used for the transesterification. The washing water may be separated from the FAME by centrifuge and this separation may be conducted after a small holding time of some 5 minutes, but a much longer holding time of 1 days to 3 days may also be applied to encourage sterol glucosides to crystallise.

According to a further embodiment of the present invention, the FAME that have been subjected to a water washing treatment may be sent to intermediate storage and then may be subjected to a further water washing treatment including liquid adsorbent. The intermediate storage causes the FAME-phase to cool down and this promotes the crystallisation of sterol glucosides and the presence of the liquid adsorbent in the water used to wash the FAME facilitates the removal of the sterol glucosides. There is no need to maintain the reduced temperature during the process according to the invention. It may be increased again to reduce the FAME viscosity and thereby increase the capacity of the centrifugal separator, there being no risk of the glucosides re-dissolving.

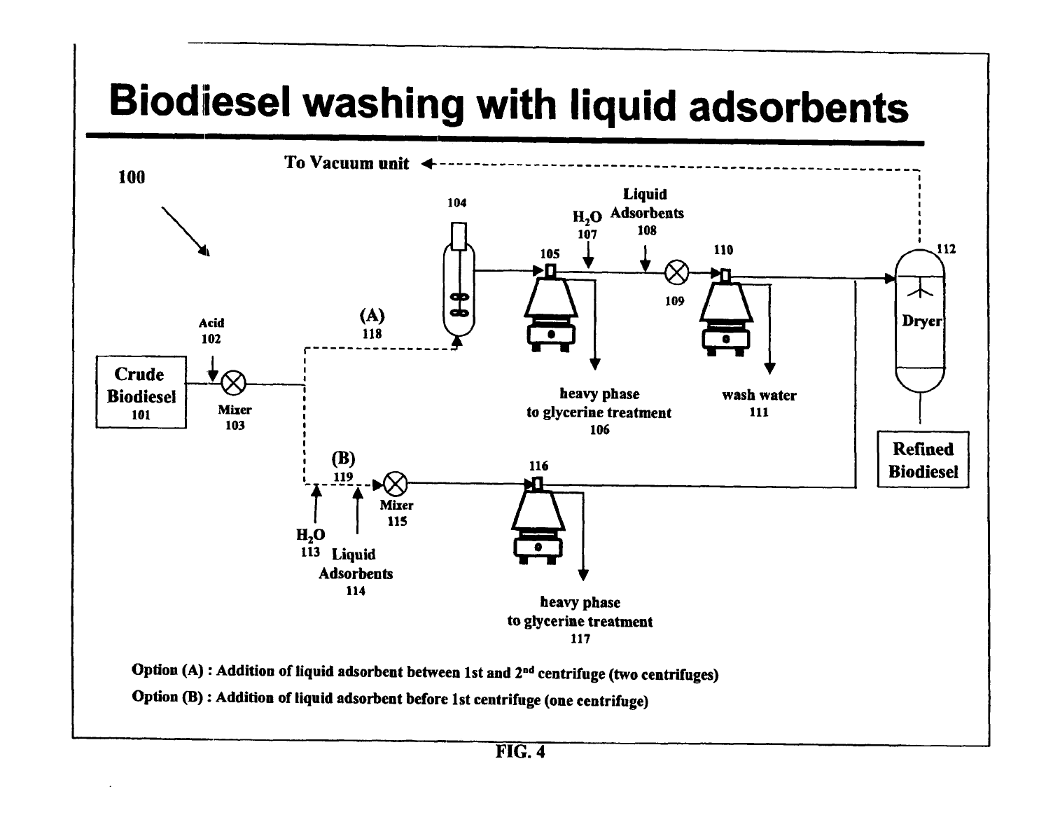

FIG. 4 depicts an apparatus 100 according to the present invention for the physical or chemical refining of fatty material, such as biodiesel. Crude biodiesel 101 is mixed with an acid 102 using an in-line mixer 103, which is then fed into tank 104 and subsequently mixed thoroughly (e.g., for 15 minutes) whereby a heavy phase including glycerol and a light phase including the fatty acid esters are created. This mixture is then sent to a first centrifuge 105 where the heavy phase 106 is separated from the light phase and then further treated to recover glycerine. The light phase including the fatty acid esters is then combined with liquid adsorbent 108 and water 107 using an in-line mixer 109, which creates an aqueous phase and a fatty acid ester phase. The liquid adsorbent may also be added to the crude biodiesel anytime prior to the first centrifuge. This mixture is sent to a second centrifuge 110 where the aqueous phase 111 is separated from the fatty acid ester phase. Subsequently, the fatty acid ester phase is sent to a dryer 112. Optionally, the crude biodiesel mixture from mixer 103 may be combined with liquid adsorbent 114 and water 113 using an in-line mixer 115, which creates an aqueous phase and a fatty acid ester phase.

Various liquid adsorbents may be utilized in the present invention, such as, sols or colloids of metal oxides, etc., and derivatives or mixtures thereof. Preferably, the liquid adsorbents include sols or colloids of metal oxides, such as for example, colloidal silica, colloidal alumina, colloidal zirconia, colloidal mania, etc. or mixtures thereof. Such materials may have a variety of particle sizes, shapes, distributions, porosity, solid content concentrations, surface coating, counter-ions, etc. There exists in a number of different commercially available grades and the particles may have a negative charge and a positive counter ion such as a sodium or ammonium cation or may have a positive charge when the stabiliser counter ion is a negative anion such as for example a chloride anion. If the fatty material that is to be treated in accordance with the process of the invention contains free fatty acids, these acids must be prevented from interfering with the removal of the impurities. Accordingly a liquid adsorbent that is negatively charged and therefore repels the free fatty acids is preferably used.

Liquid adsorbent may be in the form of a suspension in water and the solids content of such suspensions generally varies between 25% and 50% by weight. Liquid adsorbent may be prepared by any method well known from the person skilled in the art. For cost reasons, the amount of liquid adsorbent to be mixed with the fatty material in step b) of the process of the invention is preferably kept as low as possible. In practice, the amounts of liquid adsorbent used fall within the range of about 0.1 to about 10.0% by weight, preferably from about 0.2 to about 5.0% by weight; more preferably from about 0.3 to about 1.0% by weight, and even more preferably from about 0.4 to 0.6% by weight based on the total weight of the fatty material.

For the effective removal of phosphatides, a minimum contact time between the liquid adsorbent and the fatty material is preferred. This time is not critical and may range from about 1 minute to about 300 minutes, preferably from about 2 minutes to about 60 minutes, and more preferably from about 5 to about 30 minutes. Shorter times still lead to the removal of impurities such as phosphatides but will remove fewer amounts impurities and to attain the same extent of removal in a short time, more silica is required and thus the process becomes less economical. Extending the time to more than 10 minutes or even longer hardly increases the removal of impurities but has no adverse effect either.

The water optionally added in accordance with step c) of the process of the present invention serves the purpose of diluting the liquid adsorbent so that it can be more readily separated from the fatty material in step d) of the process according to the invention. If water is present, its amount is not critical. Typically, an adequate amount of water may be less than about 5% by weight, preferably less than about 3% by weight, and more preferably less than about 2% by weight of the fatty material being treated. More than 5% by weight water may be utilized but it increases the amount of wastewater to be treated and/or disposed of, and consequently the purification cost as a whole.

For the separation of the aqueous phase comprising the liquid adsorbent in accordance with step d) of the process of the present invention, a centrifugal separator is preferably used. Consequently, retrofitting existing degumming and neutralisation lines to enable them to operate the process according to the invention is often quite simple and straightforward. The centrifugal separator used to wash the degummed or neutralised oil can then be used for the removal of the liquid adsorbent. The amount of fatty material removed during this process is quite low, typically less than about 10% by weight, preferably less than about 9% by weight, more preferably less than about 8% by weight, and even more preferably less than about 7% by weight based upon the total weight of the fatty material.

Moreover, such existing lines can also operate an embodiment of the process according to the invention in which the liquid adsorbent is added to the oil stream after the caustic soda has been added to this stream and just before it enters the first centrifugal separator. In said embodiments, the spent liquid adsorbent forms part of the stream of gums (degumming) or soaps (neutralisation) respectively so that no separate silica stream has to be handled. Moreover, the oil has already been exposed to some liquid adsorbent so that somewhat less fresh liquid adsorbent has to be added in step b) of the process according to the invention to obtain the same final results.

The treated fatty material obtained after step d) contains small amounts of impurities. For example, the residual phosphorus content may be less than about 20 ppm, preferably less about 10 ppm, more preferably less than about 8 ppm, and even more preferably less than about 6 ppm. The amount of soaps present in the fatty material after step d) may be less than about 100 ppm, preferably less than about 80 ppm, more preferably less than about 60 ppm, and even more preferably less than about 50 ppm. The amount of sterol glucosides present in the fatty material after step d) may be less than about 100 ppm, preferably less than about 80 ppm, more preferably less than about 60 ppm, and even more preferably less than about 50 ppm. The use of the liquid adsorbent of the present invention also reduces haze and improves the cold soak test results of FAAE materials.

### EXAMPLES

The following Examples are given as specific illustrations of the claimed invention. It should be understood, however, that the invention is not limited to the specific details set forth in the Examples. All parts and percentages in the Examples, as well as in the remainder of the specification, are by weight unless otherwise specified.

### Example 1

Water degummed soya bean oil with a free fatty acid content of 1.38 wt % (expressed as oleic acid) and a residual phosphorus content of 144 ppm is subjected to an acid refining process involving the addition of 0.40% (volume/weight) of a 25% by weight citric acid solution, followed by the addition of 0.53% (volume/weight) of an 8% by weight caustic soda solution and of further water to a total amount corresponding to 3% (volume/weight). The gums formed during the above acid refining treatment are removed by centrifugation and the phosphorus content of the non-washed oil is determined. It had fallen to 14 ppm.

The non-washed oil is split into two parts and one part is washed with 10% water, which treatment lowered the residual phosphorus content to 8.8 ppm. Using less water would have resulted in a higher residual phosphorus content. The other part is treated with colloidal silica according to an embodiment of the present invention. An amount of 1.0% by weight of Ludox® PW-50 (Grace Davison, Grace GmbH & Co KG, Worms, Germany) is added to the oil at ambient temperature. This amount corresponds to 0.5% by weight on a dry basis. Subsequently, 2% by weight of water is added and then removed by centrifugation. The residual phosphorus content is 5.6 ppm showing that this type of colloidal silica is more effective in lowering the residual phosphorus content than washing with just water.

In Example 1, replacing the Ludox® PW-50 (pH=10.2) by the same amount of Ludox® TMA (pH=7) led to a residual phosphorus content of 7.4 ppm.

### Example 2

The same water degummed soya bean oil used in Example 1 is again acid refined using the same amounts and concentrations of citric acid and caustic soda and the same amount of total water. This led to a residual phosphorus content of 11 ppm in the non-washed oil. Because the acid treatment of the water degummed oil leads to the decomposition of the non-hydratable phosphatides still present in the water degummed oil and thus to the formation of phosphatidic acid, the non-washed oil is given a mild caustic wash to determine to what extent the use of caustic might promote the reduction of the phosphorus content of the oil.

Accordingly, 0.032% (volume/weight) of a 14% solution of caustic soda is diluted to a total volume of 3.0% of the oil and used to wash the non-washed, acid refined oil. This caused its phosphorus content to be lowered from 11 ppm to 4.3 ppm. Using the same amount of caustic solution and diluting this to a total volume of only 2.0% of the oil but including 1% of Ludox® PW-50 had a slightly better effect than operating without the colloidal silica and more water in that the residual phosphorus content is 3.7 ppm.

As can be seen from the examples, the amounts of impurities present in the fatty material treated with liquid adsorbent according to the present invention are significantly lower. Moreover, since the amount of water utilized in the treatment of the fatty material is significantly reduced, or eliminated altogether, the need for removing significant amounts of water is also eliminated, resulting in significant cost savings.

While the invention has been described with a limited number of embodiments, these specific embodiments are not intended to limit the scope of the invention as otherwise described and claimed herein. It may be evident to those of ordinary skill in the art upon review of the exemplary embodiments herein that further modifications, equivalents, and variations are possible. All parts and percentages in the examples, as well as in the remainder of the specification, are by weight unless otherwise specified. Further, any range of numbers recited in the specification or claims, such as that representing a particular set of properties, units of measure, conditions, physical states or percentages, is intended to literally incorporate expressly herein by reference or otherwise, any number falling within such range, including any subset of numbers within any range so recited. For example, whenever a numerical range with a lower limit, R_{L}, and an upper limit R_{U}, is disclosed, any number R falling within the range is specifically disclosed. In particular, the following numbers R within the range are specifically disclosed: R=R_{L}+k(R_{U}−R_{L}), where k is a variable ranging from 1% to 100% with a 1% increment, e.g., k is 1%, 2%, 3%, 4%, 5%. . . . 50%, 51%, 52%. . . . 95%, 96%, 97%, 98%, 99%, or 100%. Moreover, any numerical range represented by any two values of R, as calculated above is also specifically disclosed. Any modifications of the invention, in addition to those shown and described herein, will become apparent to those skilled in the art from the foregoing description and accompanying drawings. Such modifications are intended to fall within the scope of the appended claims. All publications cited herein are incorporated by reference in their entirety.

What is claimed is:

1. A process for the removal of impurities from fatty materials comprising the steps of:
a) providing a fatty material that may already have undergone some purification treatment;
b) adding an aqueous suspension of liquid adsorbent to said fatty material and causing it to form a mixture with said fatty material;
c) optionally adding some water to said mixture;
d) forming an aqueous phase separate from the fatty material and removing the aqueous phase from the fatty material.

2. A process according to claim 1 in which the fatty material is a crude triglyceride oil that has already been subjected to a degumming treatment.

3. A process according to claim 2 in which said degumming treatment is an acid refining treatment.

4. A process according to claim 3 in which the aqueous phase separated from the acid refined oil in step d) is added to the oil during the acid refining treatment after the addition of the caustic and before the gum separation stage.

5. A process according to claim 1 in which the fatty material is a triglyceride oil that has already undergone an alkaline neutralisation treatment.

6. A process according to claim 5 in which the aqueous phase separated from the neutralised oil in step d) is added to the oil during the alkali refining treatment after the addition of the caustic and before the soapstock separation stage.

7. A process according to claim 1 in which the fatty material is a fatty acid methyl ester.

8. A process according to claim 1 in which the amount of liquid adsorbent falls within the range of 0.2 to 1.0% by weight of fatty material.

9. A process according to claim 1 in which the water added in step c) is less than 3% by weight of the fatty material.

10. A process according to claim 1 in which the removal of the aqueous phase from the fatty material in step d) employs a centrifugal separator.

11. A process according to claim 1 in which a period of from about 1 to about 3 days elapses between steps c) and d).

12. A process according to claim 1 in which the liquid adsorbent removes phosphatides, soaps, and sterol glucosides from the treated fatty materials.

13. A process according to claim 1 in which the fatty materials comprise FAAE and the liquid adsorbent reduces the total contamination of the treated FAAE.

14. A process according to claim 1 in which the fatty materials comprise FAAE and the liquid adsorbent reduces the tendency of the treated FAAE to develop a haze.

15. A process according to claim 1 in which the fatty materials comprise FAAE and the liquid adsorbent improves the cold soak test of the treated FAAE.

16. A process according to claim 1 in which the removal process results in less than about 10% by weight of fatty material loss.

17. A process according to claim 1 in which the fatty material contains less than 10 ppm of phosphorus after step d).

18. A product made by the process according to claim 1.

19. An apparatus for performing the process according to claim 1.

20. A fatty material composition comprising;
a) fatty material that may already have undergone some purification treatment;
b) liquid adsorbent; and
c) water.

21. A fatty material composition according to claim 20 in which said liquid adsorbent is present in an amount of less than about 2% by weight based on the weight of the composition.

22. A fatty material composition according to claim 20 in which the composition contains less than 80 ppm of soaps after said liquid adsorbent is removed from the composition.

23. A fatty material composition according to claim 20 in which the composition contains less than 50 ppm of soaps after said liquid adsorbent is removed from the composition.

24. A fatty material composition according to claim 20 in which the composition contains less than 100 ppm of sterol glucosides after said liquid adsorbent is removed from the composition.

25. A fatty material composition according to claim 20 in which the composition contains less than 50 ppm of sterol glucosides after said liquid adsorbent is removed from the composition.

26. A fatty material composition according to claim 20 in which the composition contains less than 10 ppm of phosphorus after said liquid adsorbent is removed from the composition.

27. A fatty material composition according to claim 20 in which the composition contains less than 5 ppm of phosphorus after said liquid adsorbent is removed from the composition.

28. An apparatus suitable for removing impurities from fatty material comprising,
a) a first mixing device that is suitable for mixing the fatty material with an acid to form solid impurities and fatty material;
b) a first separating device in-line with the first mixing device that is suitable for removing the solid impurities from the fatty material;
c) a second mixing device in-line with the first separating device that is suitable for mixing liquid adsorbent with the fatty material; and
d) a second separating device in-line with the second mixing device that is suitable for removing the liquid adsorbent from the fatty material.

29. An apparatus according to claim 28 further comprising, a conduit connecting an outlet of the second separating device to an inlet of the first separating device that is suitable for recycling the liquid adsorbent from the second separating device to the first separating device.

30. An apparatus suitable for removing impurities from fatty material comprising,
a) a mixing device that is suitable for mixing liquid adsorbent with the fatty material; and
b) a separating device in-line with the mixing device that is suitable for removing the liquid adsorbent from the fatty material.
